(19) United States
(12) Patent Application Publication
(10) Pub. No.: US 2019/0015628 A1
(43) Pub. Date: Jan. 17, 2019

(54) CATHETER

(71) Applicant: TERUMO KABUSHIKI KAISHA, Tokyo (JP)

(72) Inventor: Michihiro Kawaguchi, Shizuoka (JP)

(73) Assignee: TERUMO KABUSHIKI KAISHA, Tokyo (JP)

(21) Appl. No.: 16/132,569

(22) Filed: Sep. 17, 2018

Related U.S. Application Data
(63) Continuation of application No. PCT/JP2017/000484, filed on Jan. 10, 2017.

(30) Foreign Application Priority Data
Mar. 18, 2016 (JP) 2016-055354

(51) Int. Cl. A61M 25/00

(52) U.S. Cl. CPC A61M 25/0041; A61M 25/0023; A61M 25/0054; A61M 2025/0037; A61M 25/0068

(57) ABSTRACT

A catheter is disclosed, which includes a flexible catheter tube. The catheter tube has an elongated main body portion, and a helical portion which is formed on a distal side of the main body portion and which is wound in a helical shape in a natural state where no external force is applied thereto. In the natural state, in the helical portion, a central axis serving as a winding center of the helical portion intersects a central axis extending along a longitudinal direction of the main body portion. In addition, it is preferable that an angle formed between the central axis of the helical portion and the central axis of the main body portion is a right angle or an obtuse angle.

### CROSS-REFERENCES TO RELATED APPLICATIONS

This application is a continuation of International Application No. PCT/JP2017/000484 filed on Jan. 10, 2017, which claims priority to Japanese Application No. 2016-055354 filed on Mar. 18, 2016, the entire contents of both of which is incorporated herein by reference.

### TECHNICAL FIELD

The present disclosure generally relates to a catheter.

### BACKGROUND ART

In recent years, vascular lesion treatment using a catheter has been actively introduced since the treatment allows relatively minimal surgical invasion. The medical procedure for vascular lesion treatment requires a procedure having excellent operability which can quickly, reliably, and selectively insert the catheter into a blood vessel system having a thin and complicated pattern.

A known catheter for use in such medical procedure includes a flexible catheter tube serving as a catheter body. The catheter tube has an elongated main body portion and a helically formed portion having a helical shape in a natural state where no external force is applied to the helical shape (for example, refer to JP-A-2001-46505). According to the catheter disclosed in JP-A-2001-46505, in the natural state, a central axis of the main body portion and a central axis serving as a winding center of the helically formed portion are located coaxially with each other. For example, depending on a shape of the blood vessel, the catheter having this configuration has a possibility that the following problems may arise.

As the shape of the blood vessel, the blood vessel has a main vessel and a side branch vessel bifurcated from the main vessel. In a case where an angle formed between the main vessel and the side branch vessel is an obtuse angle, for example, in a range of 120 degrees to 180 degrees, if the catheter needs to be inserted into the side branch vessel from the main vessel, a distal end of the helically formed portion may not be oriented toward the side branch vessel. Consequently, the helically formed portion cannot be inserted into the side branch vessel. In addition, a problem may arise in that the main body portion is inevitably deflected.

In addition, in another catheter, a distal portion is bent at one location in the natural state. In a case of this catheter, if the catheter needs to be inserted into the side branch vessel from the main vessel, the catheter comes into contact with one or two locations of the main vessel or the side branch vessel. In a case where the distal portion of the catheter is pushed into the side branch vessel by setting the contact point as a fulcrum to the side branch vessel, the fulcrum is located at only one or two points within one plane, and the number of the fulcrums sufficient for pushing the distal portion of the catheter may not be secured. Consequently, a problem arises in that the distal portion of the catheter is less likely to be pushed or in that the catheter falls out of the side branch vessel.

### SUMMARY

A catheter is disclosed which has excellent pushing ability when the catheter is pushed (or advanced) into a side branch vessel from a main vessel in a blood vessel.

The catheter disclosed here may involve the following aspects (1) to (10).

(1) A catheter is disclosed, which includes a flexible catheter tube, in which the catheter tube has an elongated main body portion, and a helical portion which is formed on a distal side of the main body portion and which is wound in a helical shape in a natural state where no external force is applied to the helical shape. In the natural state, in the helical portion, a central axis serving as a winding center of the helical portion intersects a central axis extending along a longitudinal direction of the main body portion.

(2) In the catheter according to (1) described above, an angle formed between the central axis of the helical portion and the central axis of the main body portion is a right angle or an obtuse angle.

(3) In the catheter according to (2) described above, a size of the obtuse angle is 90 degrees to 120 degrees.

(4) In the catheter according to any one of (1) to (3) described above, the number of winding times of the helical portion is once to three times.

(5) In the catheter according to any one of (1) to (4) described above, in the natural state, an outer diameter of the helical portion gradually increases toward the distal side of the main body portion.

(6) In the catheter according to (5) described above, in the natural state, a pitch of the helical portion is three times to six times an average outer diameter of the helical portion.

(7) In the catheter according to any one of (1) to (6) described above, the catheter tube has an opening portion which is open on a distal end of the catheter tube. In the natural state, the opening portion is located at a position which is different from a position on the central axis of the main body portion.

(8) In the catheter according to any one of (1) to (7) described above, the catheter tube has an opening portion which is open on a distal end of the catheter tube. In the natural state, the opening portion faces a winding direction of the helical portion.

(9) In the catheter according to any one of (1) to (8) described above, the catheter is used by inserting a guide wire into the catheter tube. The helical portion maintains the helical shape even in a state where the guide wire is inserted into the catheter tube.

(10) In the catheter according to any one of (1) to (9) described above, the helical portion has rigidity which is lower than rigidity of the main body portion.

According to the present disclosure, when the catheter is pushed (or advanced) into the side branch vessel from the main vessel in the blood vessel, it is possible to secure at least three fulcrums which are not located within the same plane inside the blood vessel. In this manner, the catheter can be sufficiently and reliably pushed (or advanced) into the side branch vessel, and the catheter can exhibit excellent pushing ability.

In accordance with an aspect, a catheter is disclosed comprising: a flexible catheter tube, the catheter tube having an elongated main body portion and a helical portion which is formed on a distal side of the main body portion, the helical portion being wound in a helical shape in a natural state where no external force is applied to the helical portion; each of the main body portion and the helical portion of the catheter tube having an inner layer and an outer layer, the inner layer and the outer layer of the helical portion having a Shore hardness with is less than a Shore hardness of the inner layer and outer layer of the main body portion; and wherein in the natural state, in the helical portion, a central axis serving as a winding center of the helical portion intersects a central axis extending along a longitudinal direction of the main body portion.

In accordance with another aspect, a method is disclosed comprising: inserting a flexible catheter tube into a main vessel, the catheter tube having an elongated main body portion and a helical portion which is formed on a distal side of the main body portion, the helical portion being wound in a helical shape in a natural state where no external force is applied to the helical portion, and in the helical portion in the natural state, a central axis serving as a winding center of the helical portion intersects a central axis extending along a longitudinal direction of the main body portion; inserting a guide wire into the flexible catheter tube and pushing the catheter tube into the main vessel along with the guide wire; and guiding a distal portion of the helical portion of the catheter tube into a side branch vessel, the side branch vessel being bifurcated from the main vessel.

### BRIEF DESCRIPTION OF THE DRAWINGS

FIG. 1 is a plane view illustrating an embodiment of a catheter according to the present disclosure.

FIG. 2 is a front view when viewed in a direction of an arrow A illustrated in FIG. 1.

FIG. 3 is a longitudinal sectional view of the catheter illustrated in FIG. 1.

FIG. 4 is a perspective view illustrating a use state of the catheter illustrated in FIG. 1.

FIG. 5 is a schematic view of a cross section taken along line B-B in FIG. 4.

FIG. 6 is a schematic plane view when viewed in a direction of an arrow C illustrated in FIG. 4.

FIG. 7 is a schematic plane view illustrating a state where the catheter in a state illustrated in FIG. 6 is further pushed forward in a direction toward a distal end.

### DESCRIPTION OF EMBODIMENTS

Set forth below with reference to the accompanying drawings is a detailed description of embodiments of a catheter representing examples of the inventive catheter disclosed here. In some cases, a dimension ratio in the drawings may be exaggerated and different from a ratio used in practice in order to facilitate the description.

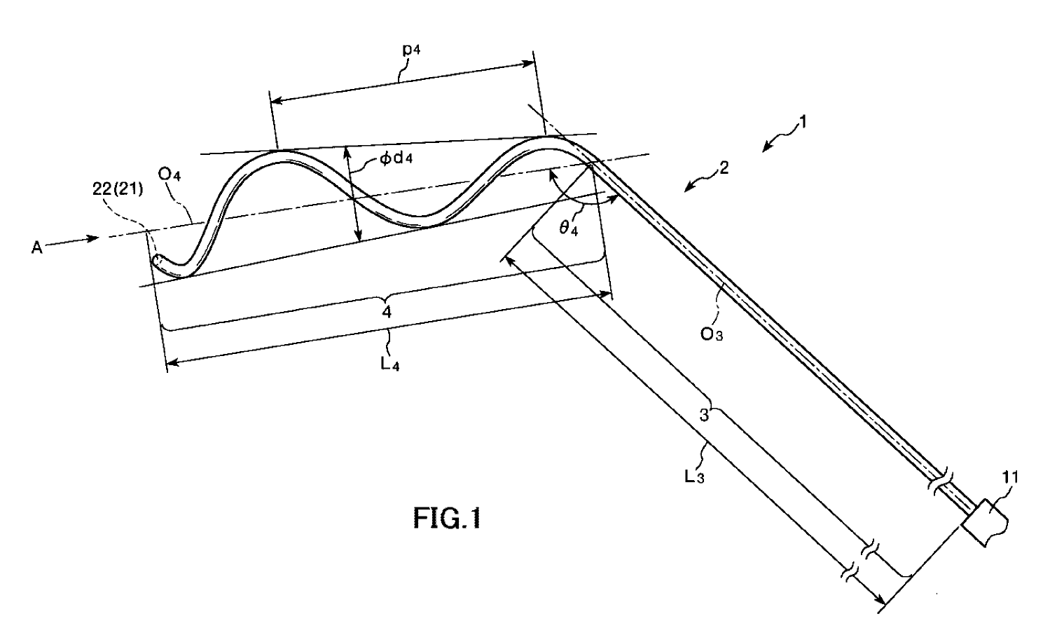

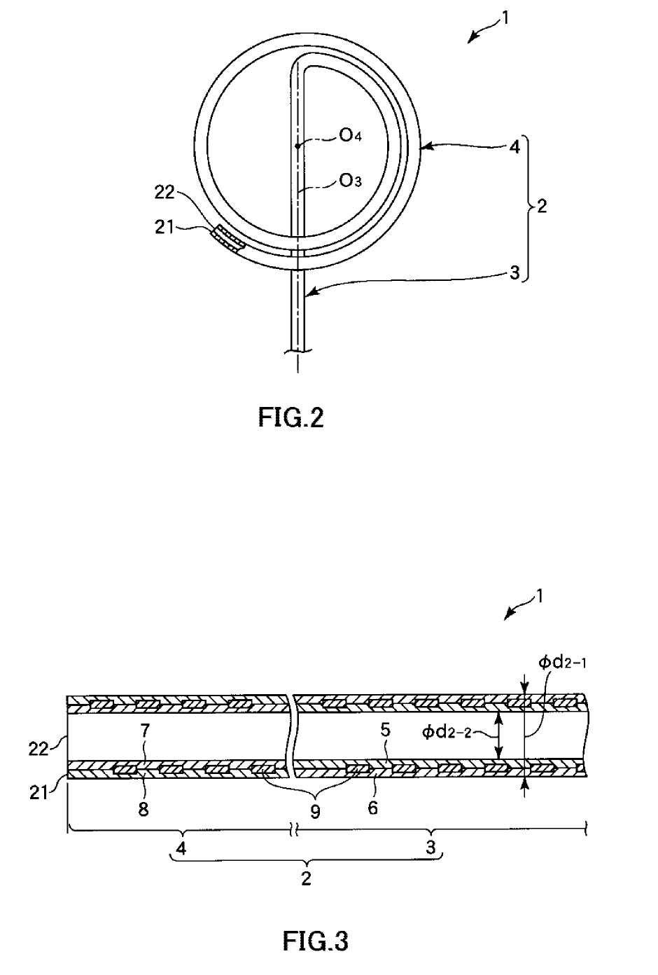

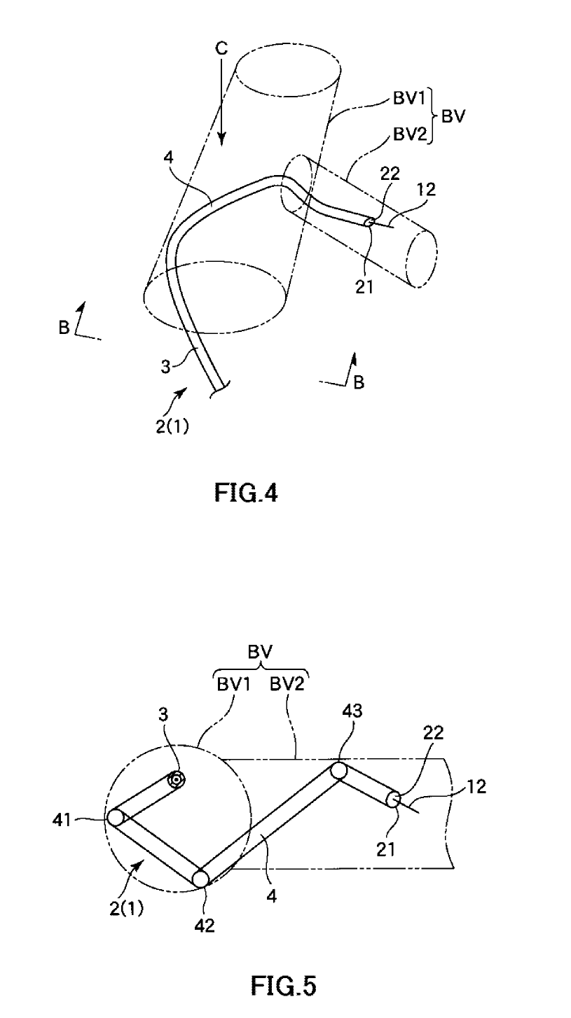

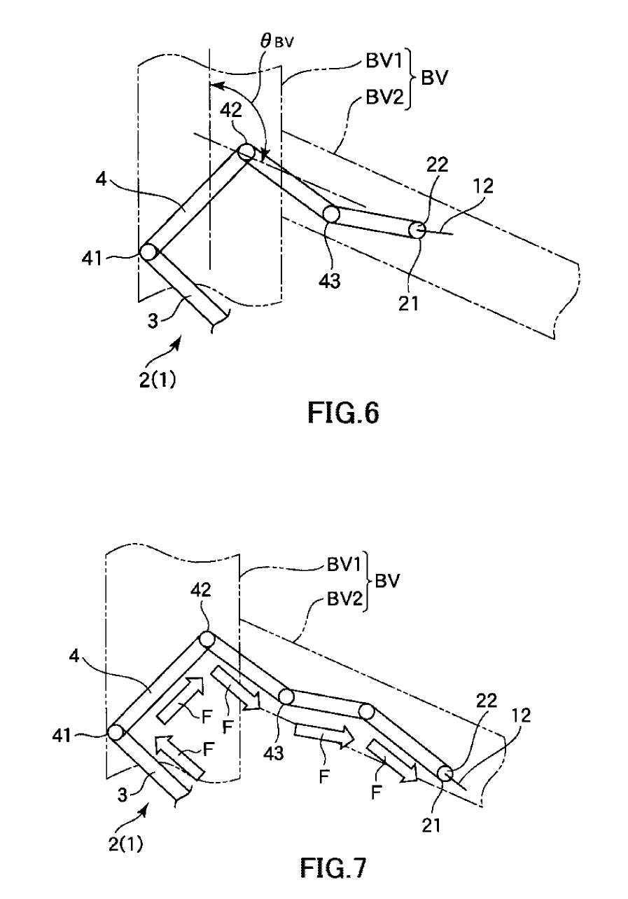

FIG. 1 is a plane view illustrating the embodiment of the catheter according to the present invention. FIG. 2 is a front view when viewed in a direction of an arrow A illustrated in FIG. 1. FIG. 3 is a longitudinal sectional view of the catheter illustrated in FIG. 1. FIG. 4 is a perspective view illustrating a use state of the catheter illustrated in FIG. 1. FIG. 5 is a schematic view of a cross section taken along line B-B in FIG. 4. FIG. 6 is a schematic plane view when viewed in a direction of an arrow C illustrated in FIG. 4. FIG. 7 is a schematic plane view illustrating a state where the catheter in a state illustrated in FIG. 6 is further pushed forward in a direction toward a distal end. In FIG. 3, for simple understanding, a helical shape in a helical portion is omitted in the illustration, and is drawn in a straight line. Hereinafter, for the convenience of description, an operator's hand side when the catheter is gripped and used will be referred to as a “proximal end” or “proximal side”, and a side opposite to the proximal side (i.e., a side inserted into a living body) will be referred to as a “distal end” or “distal side”.

A catheter 1 illustrated in FIG. 1 can be a micro catheter including a flexible catheter tube 2 and a hub 11 disposed in a proximal portion of the catheter tube 2.

The hub 11 can serve as a grip portion gripped by the operator when the catheter tube 2 is inserted into a blood vessel BV. In addition, the hub 11 is configured to include a rigid tubular body, and communicates with the catheter tube 2. In this manner, a guide wire 12 can be inserted into the catheter tube 2 via the hub 11. Then, in this inserted state, the catheter 1 can be used as illustrated in FIGS. 4 to 7.

In a natural state of the guide wire 12, a distal portion may be straight, or may be bent or curved at one location. In a case where the distal portion is bent or curved, it can be preferable that a bent or curved direction is, for example, up to 90 degrees.

In addition, the catheter tube 2 has an opening portion 22 which is open to the inner diameter ϕd₂₋₂ on a distal end 21 of the catheter tube 2. In this manner, the distal portion of the guide wire 12 can protrude from the opening portion 22 of the catheter tube 2. Accordingly, inside the blood vessel BV, the catheter 1 can be pushed forward along the guide wire 12 while the guide wire 12 moves ahead (i.e., advanced forward into the blood vessel BV).

In the catheter tube 2, according to the present embodiment, an outer diameter ϕd₂₋₁ and an inner diameter ϕd₂₋₂ are constant along a longitudinal direction. However, that is non-limiting, and, for example, the catheter tube 2 may have a portion which gradually decreases in the outer diameter ϕd₂₋₁ in a direction toward the distal end. Alternatively, the catheter tube 2 may have a gradually decreasing portion in both of the outer diameter ϕd₂₋₁ and the inner diameter ϕd₂₋₂.

As illustrated in FIGS. 1 and 2, the catheter tube 2 has an elongated main body portion 3 and a helical portion 4 formed on the distal side of the main body portion 3.

The main body portion 3 occupies most of the catheter tube 2, and a total length L₃ of the main body portion 3 is not particularly limited. For example, it can be preferable that the total length of the main body portion 3, for example, is 900 mm to 1,700 mm. It is more preferable that the total length of the main body portion, for example, is 1,000 mm to 1,500 mm.

The main body portion 3 is straight in FIG. 1. However, for example, a portion of the main body portion 3 may be bent or curved.

The helical portion 4 has a three-dimensional shape in a state where no external force is applied to the helical portion 4. That is, the helical portion 4 is wound in a helical shape. Hereinafter, the state where no external force is applied to the helical portion 4 will be referred to as a “natural state”. A winding direction of the helical portion 4 is a clockwise direction in a configuration illustrated in FIG. 2. However, without being limited thereto, the winding direction may be a counterclockwise direction.

The helical portion 4 has rigidity so that the helical shape of the helical portion 4 can be maintained even in a state where the guide wire 12 is inserted into the helical portion 4 of the catheter 2.

The blood vessel BV can have a complicatedly bifurcated portion such as a hepatic artery. For example, as illustrated in FIG. 6, an angle θ_{BV} formed between a main vessel BV1 and a side branch vessel BV2, which is located ahead in a forward moving direction of the catheter 1, can be an obtuse angle in a range of 120 degrees to 180 degrees, in some cases. In this case, for example, the catheter in the known art (i.e., JP-A-2001-46505), which is curved at one location in the distal portion in the natural state, is less likely to be pushed and inserted into the side branch vessel BV2 from the main vessel BV1. The reason is as follows. During the insertion operation, a curved portion of the catheter of the known art comes into contact with only one location of the main vessel BV1 or the side branch vessel BV2, and only one contact point serves as a fulcrum for pushing the catheter into the side branch vessel BV2.

In addition, according to other known catheters, two, three, or more fulcrums for pushing the catheter into the side branch vessel BV2 are secured (or obtained). However, these fulcrums are all in an arranged state on the same plane. Then, even if the known catheter is pushed in this state of the fulcrums being arranged in the same plane, it is insufficient to switch a direction for pushing the known catheter into the side branch vessel BV2. As a result, the known catheter cannot be inserted into a deep side of the side branch vessel BV2.

Therefore, the catheter 1 adopts the following configuration. When the catheter tube 2 is inserted into the side branch vessel BV2 from the main vessel BV1, at least three fulcrums can be secured for pushing the catheter tube 2 into the side branch vessel BV2, which are not located within the same plane in the blood vessel BV. Hereinafter, this configuration will be described.

As illustrated in FIG. 1, in the natural state, a central axis O₄ serving as the winding center of the helical portion 4 intersect a central axis O₃ extending along the longitudinal direction of the main body portion 3. Then, an angle θ₄ on the proximal side which is formed between the central axis O₄ and the central axis O₃ is a right angle or the obtuse angle. For example, it can be preferable that the angle θ₄ is equal to or larger than 90 degrees and smaller than 180 degrees. It is more preferable that the angle θ₄ is, for example, equal to or larger than 90 degrees and equal to or smaller than 120 degrees.

In a state where the guide wire 12 is inserted into the catheter tube 2 having the helical portion 4 as described above, if the catheter tube 2 is pushed into the main vessel BV1 along the guide wire 12 while the guide wire 12 is moved ahead into the main blood vessel BV1, the distal portion of the helical portion 4 of the catheter tube 2 can be inserted into the side branch vessel BV2 as illustrated in FIG. 4. In this case, as illustrated in FIGS. 5 and 6, the helical portion 4 comes into contact with the blood vessel BV at three locations, and can be bought into a supported state (i.e., the helical portion can be supported within the main vessel BV1 and the side vessel BV2 at three locations). As these fulcrums, a fulcrum 41, a fulcrum 42, and a fulcrum 43 are provided sequentially from the proximal side. The fulcrum 41 is supported by the main vessel BV1. The fulcrum 42 is supported by the main vessel BV1 at a position different from that of the fulcrum 41. The fulcrum 43 is supported by the side branch vessel BV2. Therefore, the fulcrum 41, the fulcrum 42, and the fulcrum 43 are not present on the same straight line as illustrated in FIG. 6. As illustrated in FIG. 5, the fulcrums are scattered (i.e., arranged) in a circumferential direction of the blood vessel BV, that is, in an upward-downward direction in FIG. 5.

The number of the formed fulcrums is three in the present embodiment. However, in some cases, the number of fulcrums can be four or more depending on various conditions such as the thickness of the main vessel BV1 and the side branch vessel BV2 and the size of the angle θ_{BV}.

Then, if the catheter tube 2 is further pushed in a state illustrated in FIG. 4, a pushing force F of the catheter tube 2 can be transmitted to the distal end 21 of the helical portion 4 while the fulcrum 41, the fulcrum 42, and the fulcrum 43 serve as the fulcrums as illustrated in FIG. 7. In this manner, the helical portion 4 can be reliably inserted into a target site located on the deep side of the side branch vessel BV2 (i.e., further into the side branch vessel BV2).

In this way, according to the catheter 1, when the catheter tube 2 is pushed (or advanced) into the side branch vessel BV2 from the main vessel BV1, at least three fulcrums can be secured which are not located within the same plane inside the blood vessel BV. In this manner, the catheter tube 2 can be sufficiently and reliably pushed (or advanced) into the side branch vessel BV2. Therefore, the catheter 1 is excellent in pushing ability.

In addition, the number of helically winding times in the helical portion 4 is not particularly limited. For example, it is preferable that the number of helically winding times is once to three times, and it is more preferable that the number of helically winding times is once to twice. In this manner, regardless of the conditions of the blood vessel BV such as the thickness of the main vessel BV1 or the side branch vessel BV2 and the size of the angle θ_{BV}, it is possible to secure at least the three fulcrums which are not located within the same plane inside the blood vessel BV.

As illustrated in FIG. 1, it is preferable that an outer diameter φd₄ of the helical portion 4 gradually increases toward the distal side in the natural state. An average size of the outer diameter φd₄ of the helical portion 4 is not particularly limited. For example it is preferable that the average size of the outer diameter φd₄ of the helical portion 4 is 2 mm to 8 mm, and it is more preferable that the average size of the outer diameter φd₄ of the helical portion 4 is 3 mm to 5 mm. According to the present embodiment, the outer diameter φd₄ of the helical portion 4 gradually increases toward the distal side. However, the configuration is not limited thereto. For example, the outer diameter φd₄ of the helical portion 4 may gradually decrease toward the distal side, or may be constant along the central axis O₄. The gradually increasing diameter, the gradually decreasing diameter, and the constant diameter may be appropriately combined with each other.

In addition, it is preferable that a pitch p₄ of the helical portion 4 in the natural state, for example, is within a range of one time and 15 times the average of the outer diameter φd₄ of the helical portion 4, and it is more preferable that the pitch p₄ is 3 times to 6 times the average of the outer diameter φd₄ of the helical portion 4.

A total length L₄ extending along the central axis O₄ of the helical portion 4 is shorter than the total length L₃ of main For example, it is preferable that the total length L₄ extending along the central axis O₄ of the helical portion 4 is 0.5% to 3% of the total length L₃ of the main body portion 3, and it is more preferable that the total length L₄ extending along the central axis O₄ of the helical portion 4 is 1% to 2% of the total length L₃ of the main body portion 3. Alternatively, regardless of the size of the total length L₃ of the main body portion 3, it is possible, for example, to preferably set the total length L₄ extending along the central axis O₄ of the helical portion 4 to a range of 5 mm to 50 mm, and to more preferably set the total length L₄ extending along the central axis O₄ of the helical portion 4 to a range of 10 mm to 45 mm.

A configuration that satisfies the outer diameter ϕd₄, the pitch p₄, and the total length L₄ which are described above contributes to the secured fulcrums in the helical portion 4 inside the blood vessel BV.

As illustrated in FIG. 1, it is preferable that the opening portion 22 of the catheter tube 2 is located at a position different from a position on the central axis O₃ of the main body portion 3 in the natural state. In addition, as illustrated in FIG. 2, it is preferable that the opening portion 22 faces the winding direction of the helical portion 4 in the natural state.

Accordingly, when the opening portion 22 is located at a position different from a position on the central axis O₃ of the main body portion 3 and the opening portion 22 faces the winding direction of the helical portion 4 in the natural state, and the catheter tube 2 is pushed into the side branch vessel BV 2 from the main vessel BV 1, the distal end 21 having the opening portion 22 can be relatively easily and reliably inserted into the side branch vessel BV2 at an initial stage of the insertion of the distal end 21 into the side branch vessel BV2.

In addition, it is preferable that the helical portion 4 has rigidity which is lower than that of the main body portion 3. For example, it is preferable that a difference between Shore hardness D in the helical portion 4 and Shore hardness D in the main body portion 3 is 2 to 40, and it is more preferable that the difference between Shore hardness D in the helical portion 4 and Shore hardness D in the main body portion 3 is 5 to 20. In this manner, the catheter tube 2 becomes more flexible at the distal portion of the catheter tube 2. The “Shore hardness D” represents a physical property value specified in ASTM-D 2240, Standards of American Society for Testing and Materials.

In order to satisfy this relationship between high rigidity and low rigidity, the relationship can be satisfied by adopting the configuration illustrated in FIG. 3.

As illustrated in FIG. 3, a tube wall in the main body portion 3 has an inner layer 5 and an outer layer 6. A tube wall in the helical portion 4 has an inner layer 7 and an outer layer 8, which have the Shore hardness lower than the Shore hardness of the inner layer 5 and the outer layer 6. In addition, a reinforcement member 9 can be located (or arranged) between the inner layer 5 and the outer layer 6, and between the inner layer 7 and the outer layer 8.

For example, the material of the inner layer 5, the outer layer 6, the inner layer 7, and the outer layer 8 can include various thermoplastic elastomers such as a styrene, a polyolefin, a fluorine, a polyurethane, a polyester, a polyamide, a polybutadiene, a trans polyisoprene, a fluororubber, and a chlorinated polyethylene. If the materials of the inner layer 5, the outer layer 6, the inner layer 7, and the outer layer 8 are appropriately combined with each other, it is possible to satisfy the relationship between high rigidity and low rigidity.

The reinforcement member 9 reinforces the catheter tube 2. For example, the reinforcement member 9 can include, for example, a linear body or linear member, or a mesh-shaped body. The reinforcement member 9 is configured to include various metal materials or hard resin materials. In addition, in a case where the reinforcement member 9 is the linear body, in order to minimize the thickness of the tube wall in the main body portion 3 or the tube wall in the helical portion 4, the linear body can be, for example, a stainless steel wire, which is flattened (or crushed) into a relatively flat plate, for example, by machining. In this manner, the stainless steel wire is wound in a helical shape.

Alternatively, the reinforcement member may be configured to include a helical body, a combination between the helical body and the mesh-shaped body, and a resin tube having a single layer or a stacked layer.

The catheter according to the present disclosure has been described above with reference to the illustrated embodiments. However, the present disclosure is not limited to the embodiments of the catheter as illustrated. For example, each element of the catheter can be substituted with any desired element which can fulfill the same function. In addition, any desired element may be added to the catheter.

According to the present disclosure, a catheter is disclosed, which includes a flexible catheter tube. The catheter tube has an elongated main body portion, and a helical portion which is formed on a distal side of the main body portion and which is wound in a helical shape in a natural state where no external force is applied thereto. In the natural state, in the helical portion, a central axis serving as a winding center of the helical portion intersects a central axis extending along a longitudinal direction of the main body portion. Accordingly, the catheter according to the present disclosure is excellent in pushing ability when the catheter is pushed into the side branch vessel from the main vessel in the blood vessel. Therefore, the catheter according to the present disclosure has industrial applicability.

The detailed description above describes a catheter. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A catheter comprising:
a flexible catheter tube, the catheter tube having an elongated main body portion and a helical portion which is formed on a distal side of the main body portion, the helical portion being wound in a helical shape in a natural state where no external force is applied to the helical portion; and
wherein in the natural state, in the helical portion, a central axis serving as a winding center of the helical portion intersects a central axis extending along a longitudinal direction of the main body portion.

2. The catheter according to claim 1, wherein an angle formed between the central axis of the helical portion and the central axis of the main body portion is a right angle or an obtuse angle.

3. The catheter according to claim 2, wherein the obtuse angle is 90 degrees to 120 degrees.

4. The catheter according to claim 1, wherein a number of windings of the helical portion is one to three times.

5. The catheter according to claim 1, wherein in the natural state, an outer diameter of the helical portion gradually increases toward a distal side of the catheter.

6. The catheter according to claim 5, wherein in the natural state, a pitch of the helical portion is three times to six times an average outer diameter of the helical portion.

7. The catheter according to claim 1, wherein the catheter tube has an opening portion which is open on a distal end of the catheter tube, the opening portion in the natural state being located at a position which is different from a position on the central axis of the main body portion.

8. The catheter according to claim 1, wherein the catheter tube has an opening portion which is open on a distal end of the catheter tube, and the opening portion in the natural state faces a winding direction of the helical portion.

9. The catheter according to claim 1, wherein the catheter is used by inserting a guide wire into the catheter tube, and wherein the helical portion maintains the helical shape even in a state where the guide wire is inserted into the catheter tube.

10. The catheter according to claim 1, wherein a rigidity of the helical portion is lower than a rigidity of the main body portion.

11. A catheter comprising:
a flexible catheter tube, the catheter tube having an elongated main body portion and a helical portion which is formed on a distal side of the main body portion, the helical portion being wound in a helical shape in a natural state where no external force is applied to the helical portion;
each of the main body portion and the helical portion of the catheter tube having an inner layer and an outer layer, the inner layer and the outer layer of the helical portion having a Shore hardness with is less than a Shore hardness of the inner layer and outer layer of the main body portion; and
wherein in the natural state, in the helical portion, a central axis serving as a winding center of the helical portion intersects a central axis extending along a longitudinal direction of the main body portion.

12. The catheter according to claim 11, further comprising:
a reinforcement layer arranged between the inner layer and the outer layer of the main body portion.

13. The catheter according to claim 11, further comprising:
a reinforcement layer arranged between the inner layer and the outer layer of the helical portion.

14. The catheter according to claim 11, wherein an angle formed between the central axis of the helical portion and the central axis of the main body portion is a right angle or an obtuse angle.

15. The catheter according to claim 14, wherein the obtuse angle is 90 degrees to 120 degrees.

16. A method comprising:
inserting a flexible catheter tube into a main vessel, the catheter tube having an elongated main body portion and a helical portion which is formed on a distal side of the main body portion, the helical portion being wound in a helical shape in a natural state where no external force is applied to the helical portion, and in the helical portion in the natural state, a central axis serving as a winding center of the helical portion intersects a central axis extending along a longitudinal direction of the main body portion;
inserting a guide wire into the flexible catheter tube and pushing the catheter tube into the main vessel along with the guide wire; and
guiding a distal portion of the helical portion of the catheter tube into a side branch vessel, the side branch vessel being bifurcated from the main vessel.

17. The method according to claim 16, further comprising:
maintaining the helical shape of the helical portion of the catheter tube with the guide wire inserted into the catheter tube.

18. The method according to claim 16, further comprising:
advancing the guide wire into main vessel while the distal portion of the helical portion of the catheter tube is inserted into the side branch vessel.

19. The method according to claim 16, further comprising:
bringing the helical portion of the catheter tube into contact with the main vessel and the side branch vessel in at least three locations.

20. The method according to claim 16, further comprising:
advancing the distal portion of the helical portion of the catheter tube further into the side branch vessel.
